(19) United States
(12) Patent Application Publication
(10) Pub. No.: US 2006/0041183 A1
(43) Pub. Date: Feb. 23, 2006

(54) Electromechanical machine-based artificial muscles, bio-valves and related devices

(76) Inventors: Richard J. Massen, Albuquerque, NM (US); Luis M. Ortiz, Albuquerque, NM (US)

Correspondence Address: Kermit D. Lopez;Ortiz & Lopez, PLLC, P.O. Box 4484, Albuquerque, NM, 87196-4484, US

(21) Appl. No.: 11/007,457

(22) Filed: Dec. 9, 2004

Related U.S. Application Data
(63) Continuation-in-part of application No. 10/923,357, filed on Aug. 20, 2004.

(51) Int. Cl. A61M 1/12; A61F 2/08

(52) U.S. Cl. 600/16; 623/14.13; 623/13.13; 600/30; 600/37; 623/23.68

(57) ABSTRACT

A biological function assist apparatus composed an electromechanically-based system wrapped in protective coating and controlled by a controller, which also provides power to the electromechanically-based system. The electromechanically-based system can be formed as a mesh using MEMS or a larger electromechanically grid and wrapped around a failing heart, or the electromechanical system can be formed in a circle forming an artificial valve (e.g., sphincter). The electromechanically-based system can operate as a bone-muscle interface, thereby functioning in place of tendons.

### INVENTION PRIORITY

This application is a continuation-in-part of U.S. patent application Ser. No. 10/923,357, entitled “Micro electromechanical machine-based ventricular assist apparatus,” which was filed with the United States Patent and Trademark Office on Aug. 20, 2004, and which is incorporated herein by reference herein in its entirety.

### TECHNICAL FIELD

The embodiments are generally related to electro-mechanical systems. The embodiments are also related to artificial muscles. More particularly, embodiments are related to electromechanical-based artificial muscles, bio-valves and related devices. Embodiments are also related to devices for assisting natural human organs and body parts assisted by electromechanical-based devices.

### BACKGROUND OF THE INVENTION

The natural human heart and accompanying circulatory system are critical components of the human body and systematically provide the needed nutrients and oxygen for the body. As such, the proper operation of a circulatory system, and particularly, the proper operation of the heart, is critical in the overall health and well being of a person. A physical ailment or condition which compromises the normal and healthy operation of the heart can therefore be particularly critical and may result in a condition which must be medically remedied.

Specifically, the natural heart, or rather the cardiac tissue of the heart, can fail for various reasons to a point where the heart can no longer provide sufficient circulation of blood for the body so that life can be maintained. To address the problem of a failing natural heart, conventional solutions have been offered to provide techniques for which circulation of blood might be maintained.

Some solutions involve replacing the heart. Other solutions maintain the operation of the existing heart. One such solution has been to replace the existing natural heart in a patient with an artificial heart or a ventricular assist device. In utilizing artificial hearts and/or assist devices, a particular problem stems from the fact that the materials used for the interior lining of the chambers of an artificial heart are in direct contact with the circulating blood. Such contact may enhance the undesirable clotting of the blood, may cause a build-up of calcium, or may otherwise inhibit the blood's normal function. As a result, thromboembolism and hemolysis may occur.

Additionally, the lining of an artificial heart or a ventricular assist device can crack, which inhibits performance, even when the crack is at a microscopic level. Moreover, these devices must be powered by a power source, which may be cumbersome and/or external to the body. Such drawbacks have limited use of artificial heart devices to applications having too brief of a time period to provide a real lasting benefit to the patient.

An alternative procedure also involves replacement of the heart and includes transplanting the heart from another human or animal into the patient. The transplant procedure requires removing an existing organ (i.e. the natural heart) from the patient for substitution with another organ (i.e. another natural heart) from another human, or potentially, from an animal. Before replacing an existing organ with another, the substitute organ must be “matched” to the recipient, which can be, at best, difficult, time consuming and expensive to accomplish. Furthermore, even if the transplanted organ matches the recipient, a risk exists that recipient's body will still reject the transplanted organ and attack it as a foreign object. Moreover, the number of potential donor hearts is far less than the number of patients in need of a natural heart transplant. Although use of animal hearts would lessen the problem of having fewer donors than recipients, there is an enhanced concern with respect to the rejection of the animal heart.

In an effort to continue use of the existing natural heart of a patient, other attempts have been made to wrap skeletal muscle tissue around the natural heart to use as an auxiliary contraction mechanism so that the heart may pump. As currently used, skeletal muscle cannot alone typically provide sufficient and sustained pumping power for maintaining circulation of blood through the circulatory system of the body. This is especially true for those patients with severe heart failure.

Another system developed for use with an existing heart for sustaining the circulatory function and pumping action of the heart, is an external bypass system, such as a cardiopulmonary (heart-lung) machine. Typically, bypass systems of this type are complex and large, and, as such, are limited to short term use, such as in an operating room during surgery, or when maintaining the circulation of a patient while awaiting receipt of a transplant heart. The size and complexity effectively prohibit use of bypass systems as a long-term solution, as they are rarely portable devices. Furthermore, long-term use of a heart-lung machine can damage the blood cells and blood borne products, resulting in post surgical complications such as bleeding, thromboembolism function, and increased risk of infection.

Still another solution for maintaining the existing natural heart as the pumping device involves enveloping a substantial portion of the natural heart, such as the entire left and right ventricles, with a pumping device for rhythmic compression. That is, the exterior wall surfaces of the heart are contacted and the heart walls are compressed to change the volume of the heart and thereby pump blood out of the chambers. Although somewhat effective as a short-term treatment, the pumping device has not been suitable for long-term use.

Typically, with such compression devices, a vacuum pressure is needed to overcome cardiac tissue/wall stiffness, so that the heart chambers can return to their original volume and refill with blood. This “active filling” of the chambers with blood limits the ability of the pumping device to respond to the need for adjustments in the blood volume pumped through the natural heart, and can adversely affect the circulation of blood to the coronary arteries. Furthermore, natural heart valves between the chambers of the heart and leaching into and out of the heart are quite sensitive to wall and annular distortion. The movement patterns that reduce a chamber's volume and distort the heart walls may not necessarily facilitate valve closure (which can lead to valve leakage).

Therefore, mechanical pumping of the heart, such as through mechanical compression of the ventricles, must address these issues and concerns in order to establish the efficacy of long term mechanical or mechanically assisted pumping. Specifically, the ventricles must rapidly and passively refill at low physiologic pressures, and the valve functions must be physiologically adequate. The mechanical device also must not impair the myocardial blood flow of the heart. Still further, the left and right ventricle pressure independence must be maintained within the heart.

Another major obstacle with long term use of such pumping devices is the deleterious effect of forceful contact of different parts of the living internal heart surface (endocardium), one against another, due to lack of precise control of wall actuation. In certain cases, this cooptation of endocardium tissue is probably necessary for a device that encompasses both ventricles to produce independent output pressures from the left and right ventricles. However, it can compromise the integrity of the living endothelium.

Mechanical ventricular wall actuation has shown promise, despite the issues noted above. As such, devices have been invented for mechanically assisting the pumping function of the heart, and specifically for externally actuating a heart wall, such as a ventricular wall, to assist in such pumping functions.

One particular type of mechanical ventricular actuation device that has been developed is a Left Ventricular Assist Device (LVAD), which is designed to support the failing heart. Such a device must augment systolic function. Diastolic function must also be augments or at the very least, not worsened, while allowing blood flow between the right and left ventricular portions of the heart. If the LVAD relies on a pump mechanism, the heart must still be able to beat 45 to 40 million times per year. The LVAD must therefore be durable and should function flawlessly or permit some degree of cardiac function in case of device failure. Such devices and/or systems must also permit a minimal risk for blood clot production and should be resistant to infection.

Other bodily functions rely on physical manipulation of muscles. For example, urinary and anorhectal sphincter valves control incontinence when operating properly. Sphincter valves are also founding the digestive tract where food passes from the esophagus into the stomach. Sphincter valves, however, tend to malfunction or lose range of operation. For example, after childbirth or as the human body ages. Surgery will sometimes correct incontinence in patients or reduce occurrences of Gastro esophageal reflux disease (GERD). Unfavorable conditions, however, often return or are sometimes not correctable using current treatments. Current artificial sphincter prototypes are composed of elastic and inflated with air. Erosion, probably from continuous high tonic pressure of inflated balloon in the urinary tract, can lead to infection and device failure. Therefore, there is a need for artificial means of restoring sphincter valve operation for digestive conditions. It is the inventors' belief that sphincter valve operation can be assisted or replaced using electromechanical systems.

Tendons are the thick fibrous cords that attach muscles to bone. They function to transmit the power generated by a muscle contraction to move a bone. Use of tendons can fail following trauma or because of arthritis. It is the inventors' belief that the movement of hands, fingers, arms and legs that lose mobility can be assisted using electromechanical systems.

It is believed by the present inventors that a solution to the aforementioned problems associated with conventional ventricular assist devices and sphincter valves involves the use of electromechanical systems, such as mini-machines and so-called micro electromechanical system (MEMS) technology. It is also believed that electromechanical systems can offer alternatives to other muscular dysfunctions encountered by patients due to age, disease or accidental causes.

“MEMS” is an abbreviation for Micro Electro Mechanical Systems. This is a rapidly emerging technology combining electrical, electronic, mechanical, optical, material, chemical, and fluids engineering disciplines. As the smallest commercially produced “machines”, MEMS devices are similar to traditional sensors and actuators although much, much smaller, e.g. complete systems are typically a few millimeters across, with individual features/devices of the order of 1-100 micrometers across. MEMS devices are manufactured either using processes based on Integrated Circuit fabrication techniques and materials, or using new emerging fabrication technologies such as micro injection molding.

These former processes involve building the device up layer by layer, involving several material depositions and etch steps. A typical MEMS fabrication technology may have a 5 step process. Due to the limitations of this “traditional IC” manufacturing process MEMS devices are substantially planar, having very low aspect ratios (typically 5-10 micro meters thick). It is important to note that there are several evolving fabrication techniques that allow higher aspect ratios such as deep x-ray lithography, electro deposition, and micro injection molding.

MEMS devices are typically fabricated onto a substrate (chip) that may also contain the electronics required to interact with the MEMS device. Due to the small size and mass of the devices, MEMS components can be actuated electrostatically (piezoelectric and bimetallic effects can also be used). The position of MEMS components can also be sensed capacitively. Hence the MEMS electronics include electrostatic drive power supplies, capacitance charge comparators, and signal conditioning circuitry. Connection with the macroscopic world is via wire bonding and encapsulation into familiar BGA, MCM, surface mount, or leaded IC packages.

A common MEMS actuator is the “linear comb drive” shown in FIG. 1, which consists of rows of interlocking teeth; half of the teeth are attached to a fixed “beam”, the other half attach to a movable beam assembly. Both assemblies are electrically insulated. By applying the same polarity voltage to both parts the resultant electrostatic force repels the movable beam away from the fixed. Conversely, by applying opposite polarity the parts are attracted. In this manner the comb drive can be moved “in” or “out” and either DC or AC voltages can be applied. The small size of the parts (low inertial mass) means that the drive has a very fast response time compared to its macroscopic counterpart. The magnitude of electrostatic force is multiplied by the voltage or more commonly the surface area and number of teeth. Commercial comb drives have several thousand teeth, each tooth approximately 10 micro meters long. Drive voltages are CMOS levels.

### BRIEF SUMMARY OF THE INVENTION

The following summary of the invention is provided to facilitate an understanding of some of the innovative features unique to the embodiments and is not intended to be a full description. A full appreciation of the various aspects of the embodiments can be gained by taking the entire specification, claims, drawings, and abstract as a whole.

It is a feature of the embodiments to provide eletromechanical system for use to assist or replace human muscles, muscle/tendon operation, and sphincter valves.

It is another feature of the embodiments to provide an electromechanically-based ventricular assist device.

It is another feature of the embodiments to provide an electromechanically-based ventricular assist device in the form of at least one of: a cardial patch and a whole-heart wrap/jacket.

It is another feature of the embodiments to provide an electromechanically-based bio valve.

It is another feature of the embodiments to provide an electromechanically-based bio valve that can be used as at least one of: an artificial anorectal sphincter, an artificial urinary sphincter, and an artificial gastroesophageal sphincter.

It is another feature of the embodiments to provide electromechanically-based muscle and tendon operation within human extremities.

It is another feature of the embodiments to provide an electromechanically-based muscle-tendon interface.

In accordance with more features of the embodiments, a system is described that includes an electromechanical-based biological system interface, at least one sensor to monitor biological functions, a microprocessor for analyzing biological functions measured by the at least one sensor, a controller for causing operation of the electromechanical-based to operate at least one of a ventricular assist device, bio valve and muscle-tendon interface, under direction of the microprocessor.

In accordance with more features of the embodiments, a system is described that includes integrated wire network provides sensory feedback, controlled contraction or relaxation of any single actuator or actuator groups, programmable contraction or expansion, and reflexic contraction or expansion from natural internal pacemakers.

In accordance with more features of the embodiments, a system is described that includes programmable contraction and expansion of artificial muscle regions and sub-regions, or artificial valves, programmable response to stimulus, and resistance to mechanical failure since multiple components operate in parallel.

It is yet a further aspect of the embodiments to provide for a ventricular assist device and system that is composed sheet of MEMS-based material that can be wrapped around a failing heart to support ventricular activities thereof.

Additionally, each electromechanical element is linkable, contractile, durable and electrically insulated to performance characteristics by design. For example, a sheet can be configured from a flexible and/or a pliable material, and may be arranged as a sheath and/or in a mesh arrangement of the MEMS elements.

The embodiments can be used for assistance of the following bodily functions/systems: Abdominal wall substitutes; Diaphragm substitutes; Artificial muscles such as skeletal muscle, Ocular muscle, Visceral muscle; Tendons as a muscle-bone interface; conduits; Sphincter Valves associated with reservoirs, the esophagus, prostrates, and the urinary bladder.

### BRIEF DESCRIPTION OF THE DRAWINGS

The accompanying figures, in which like reference numerals refer to identical or functionally-similar elements throughout the separate views and which are incorporated in and form a part of the specification, further illustrate at least one embodiment and, together with the detailed description of the invention, serve to explain the principles of embodiments.

FIG. 1 illustrates a heart with a mesh support system surrounding it for support in accordance with one embodiment;

FIG. 2 illustrates a pictorial perspective view of a human heart whose ventricular activities can be supported and enhanced utilizing an embodiment;

FIG. 3 illustrates another pictorial perspective view of a human heart whose ventricular activities being supported and enhanced utilizing another embodiment;

FIG. 4, labeled as “prior art,” illustrates a conventional comb drive actuator;

FIG. 5 illustrates a plurality of comb drive actuators linked together in a chain-like fashion in accordance with an aspect of an embodiment;

FIG. 6 illustrates the plurality of actuators link together in a chain as shown in FIG. 4 and surrounded by a protective, flexible material;

FIG. 7 illustrates a motor including a gear having teeth that interface with complimentary teeth formed along a moveable, flexible strap;

FIG. 8 illustrates the motor-strap configuration of FIG. 6 surrounded by a protective, flexible material;

FIG. 9 illustrates a electro-mechanical system in accordance with features of the embodiments operating as a sphincter valve and including a microprocessor;

FIG. 10 illustrates another electro-mechanical system in accordance with features of the embodiments operating as a sphincter valve;

FIG. 11 illustrates a pictorial diagram of an artificial sphincter valve enabled in accordance with features of the embodiments in a “closed” position after having electro-mechanical assisted operation, and also shown is a sensor for monitoring bodily function in relation to operation of the sphincter valve;

FIG. 12 illustrates a pictorial perspective view of a human hand and arrows indicating along the human hand where an electromechnical system in accordance with features that can be incorporated to provide muscle-tendon operation assistance;

FIG. 13 illustrates a pictorial perspective view of a human digestive system and arrows pointing to locations (e.g., esophagus, rectum, urinary tract) that artificial sphincter valves in accordance with embodiments;

FIG. 14 illustrates a pictorial perspective view of a human body and arrows pointing to locations (e.g., eyes, heart, esophagus, digestive tract, arms, legs, hands, feet) wherein electro-mechanical systems in accordance with features of the present invention can be employed, e.g., in the form of sphincter valves or muscle-tendon interfaces, in accordance with an alternate embodiment;

FIG. 15 is a flow diagram illustrating steps of how an electromechanical system in accordance with features of the present invention can operate autonomously within the human body, in accordance with an alternate embodiment; and

FIG. 16 illustrates a flow diagram showing steps wherein an electro-mechanical system operates within the human body in association with some human intervention, in accordance with an alternate embodiment.

### DETAILED DESCRIPTION

The particular values and configurations discussed in these non-limiting examples can be varied and are cited merely to illustrate at least one embodiment and are not intended to limit the scope thereof.

A natural human heart includes a lower portion comprising two chambers, namely a left ventricle and a right ventricle, which function primarily to supply the main pumping forces that propel blood through the circulatory system, including the pulmonary system (lungs) and the rest of the body, respectively. Hearts also includes an upper portion having two chambers, a left atrium and a right atrium, which primarily serve as entryways to the ventricles, and also assist in moving blood into the ventricles. The interventricular wall or septum of cardiac tissue separating the left and right ventricles is defined externally by an interventricular groove on the exterior wall of the natural heart. The atrioventricular wall of cardiac tissue separating the lower ventricular region from the upper atrial region is defined by atrioventricular groove on the exterior wall of the natural heart. The configuration and function of the heart is known to those skilled in this art.

Generally, the ventricles are in fluid communication with their respective atria through an atrioventricular valve in the interior volume defined by heart. More specifically, the left ventricle is in fluid communication with the left atrium through the mitral valve, while the right ventricle is in fluid communication with the right atrium through the tricuspid valve. Generally, the ventricles are in fluid communication with the circulatory system (i.e., the pulmonary and peripheral circulatory system) through semilunar valves. More specifically, the left ventricle is in fluid communication with the aorta of the peripheral circulatory system, through the aortic valve, while the right ventricle is in fluid communication with the pulmonary artery of the pulmonary, circulatory system through the pulmonic or pulmonary valve.

The heart basically acts like a pump. The left and right ventricles are separate, but share a common wall, or septum. The left ventricle has thicker walls and pumps blood into the systemic circulation of the body. The pumping action of the left ventricle is more forceful than that of the right ventricle, and the associated pressure achieved within the left ventricle is also greater than in the right ventricle. The right ventricle pumps blood into the pulmonary circulation, including the lungs. During operation, the left ventricle fills with blood in the portion of the cardiac cycle referred to as diastole. The left ventricle then ejects any blood in the part of the cardiac cycle referred to as systole. The volume of the left ventricle is largest during diastole, and smallest during systole. The heart chambers, particularly the ventricles, change in volume during pumping. The natural heart, or rather the cardiac tissue of the heart, can fail for various reasons to a point where the heart can no longer provide sufficient circulation of blood from its operation so that bodily function and life can be sustained.

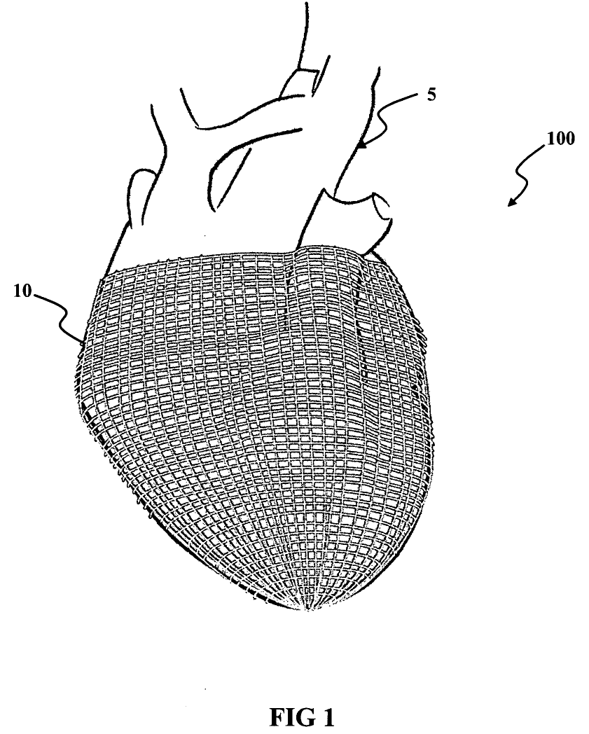

Referring to FIG. 1, a heart 5 is illustrated with a mesh support system 10 surrounding it for support in accordance with embodiment of the present invention. The mesh-like sheet can offer support to a failing heart so that it will not expand/swell, and can also include electromechanical operation within its grid-like structure (as wil be further explained) in order to assist with pumping of the heart.

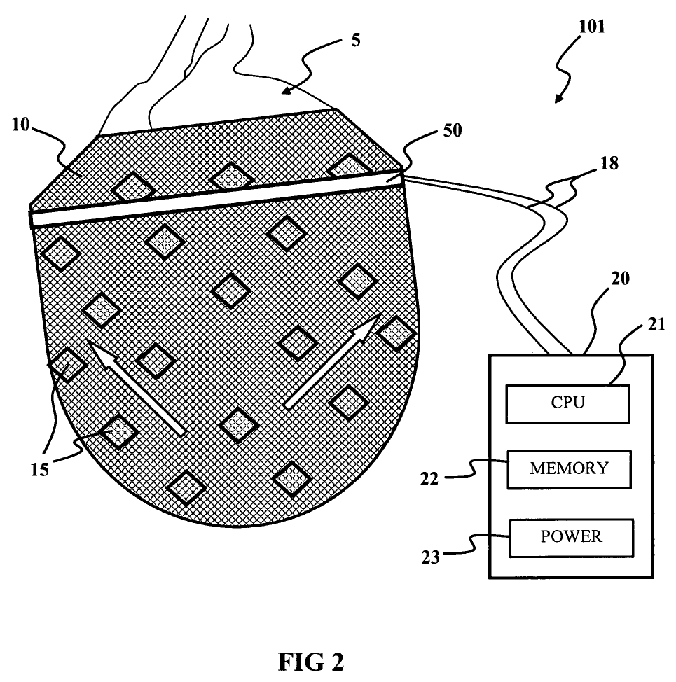

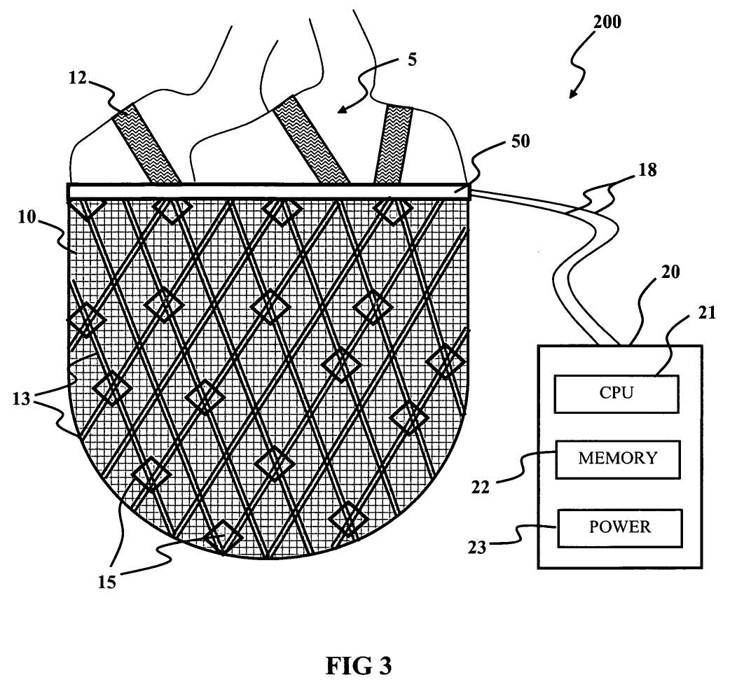

FIG. 2 illustrates a system wherein a biological function is controlled by a microprocessor and an electromechanical hardware implanted upon a biological system, in particular a human heart. The heart 5 is adapted with a mesh-like sheet of electro-mechanical material 10 of MEM-based material wrapped about the heart, in accordance with one embodiment of the present invention. Note that in FIGS. 2 and 3, identical or similar parts or elements are generally indicated by identical reference numerals. Thus, heart 5 depicted in FIG. 1 is also depicted in FIG. 2. Sheet 10 indicates wrapping of substantially all of the heart 5.

Indicated in FIG. 1 are five general requirements, including, as indicated at point 1, that the electromechanically-based material of sheet 10 is preferably composed of a group of (MEMS) elements linked to one another. As indicated by the large arrows on the mesh, each MEMS element among the group of MEMS elements forming sheet 10 can possess an embedded electrical polarity, which contributes to the generation of a force for contraction or expansion by sheet 10 in order to support natural ventricular activities of heart 5, which are believed to be similar to a wringing action by the muscles, and prevent failure thereof when sheet 10 is wrapped around heart 5.

As indicated at point 3, sheet 40 thus provides a contractile function. Relaxation can occur in the system by reversing the electrical polarity in diastole, or by allowing the heart muscles to expand into relaxed states between cycles while power is no longer applied. It should be appreciated that each electromechanical element among said plurality of elements composing sheet 10 is electrical insulated. Electrical contact can be facilitated between a controller 20 and the mesh 10 by band 50, which can operate as a conduit for electrical wires and feedback wiring 18. The wiring connects positive contacts associated with the electromechanical elements composed of the mesh 10. A common ground can be provided using the mesh material, or separate contacts to each electromechanical device can be provided; however, it can be appreciated that less wiring is needed where a common ground is provided using the mesh 10.

Also shown in FIG. 2 are sensors 15 integrated with the mesh 10. The sensor can monitor pressure created between the heart 6 and mesh 10. Results can be provided to the controller 20 where it can be analyzed by the CPU 21. The controller 20 can be provided as a self-contained module, similar to that provided with pacemakers. The controller 20 also provides power 23 to the mesh 10, sensors 15 and CPU 21. A memory 22 can be used to store results obtained from the sensor, and can also contained program instructions for the CPU 21 to use while operating the electromechanical devices integrated with the mesh 10.

FIG. 3 illustrates a system 200 for assisting operation of a natural heart in accordance with alternative embodiments. The system 200 still utilizes a controller 20, wiring 18, conduit 50 and mesh 10; however, the mesh 10 in FIGS. 1 and 2 no longer has MEMS-based electromechanical devices integrated therein. Mesh 10 operates as a support material, like stockings, for the heart to prevent it from swelling. Electromechanical devices and sensor 15 can be mounted on or next to the mesh 10. It is envisioned that mini-scaled electronmechanical devices can also be used to operate a system in accordance with the embodiments. Mini-devices can be used to cause pumping of the heart utilizing the bands 13 illustrated in FIG. 2.

The electromechanical devices can be integrated within the bands 13 or firmly along the conduit 50 wherefrom the electromechanical devices can pull on the bands 13 in order to assist the heart with pumping. It can be noted that MEMS-based device described with respect to FIG. 2 could also be mounted along the conduit 5, but it is believed MEMS would be more effective if scattered about the mesh 10 due to size and necessary torque. Sensor 15 can be deployed along the bands, between the bands 13 and the heart 5. Also shown in FIG. 3 are support straps 12, which can be utilized to provide additional support to the mesh 10 and supported components (e.g., sensors 15 and devices (not shown)). Straps, like suspenders, can support the mesh 10 around most of the heart 5 and ensure pressure is applied against the heart by the electromechanical devices via the bands 13 and mesh 10.

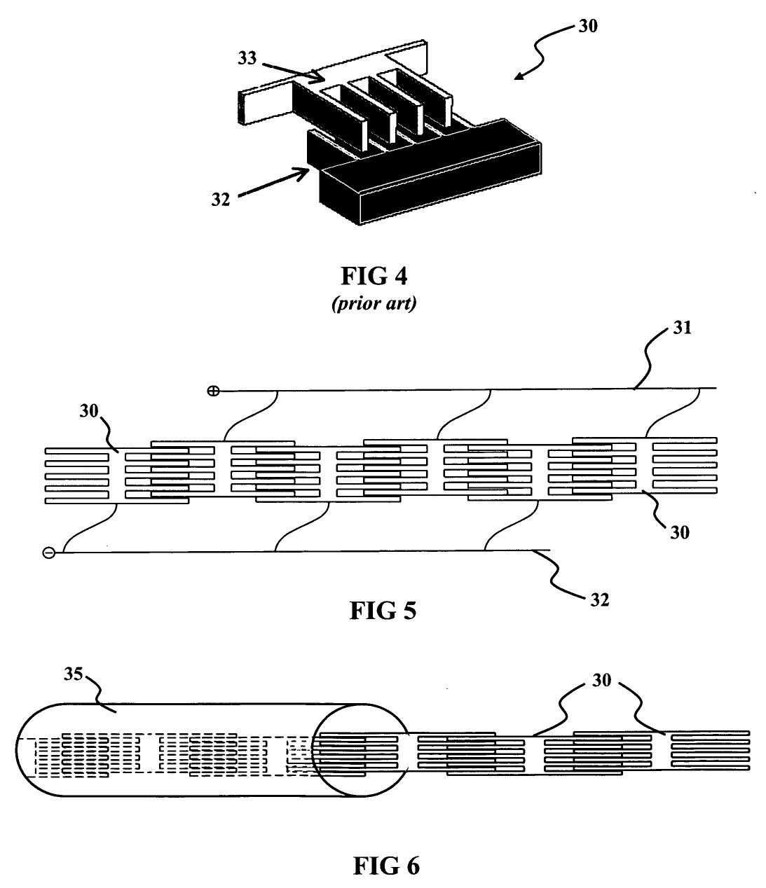

FIG. 4 is a basic prior art illustration of a comb drive actuator. Such actuators are often used in MEMS. A comb drive actuator 30 requires two components operating at different polarities to properly operate. Illustrated is a base member 32 having several teeth (similar to teeth on a comb) and a moving member 33 which also has teeth, but the moving member's teeth are complimentary to the base member's 32 teeth. During operation, electricity can be applied to each of the members 32/33 causing the members to be drawn together because of magnetic attraction between their respective teeth.

The teeth should never be allowed to touch, because a short will cause the comb drive actuator to malfunction. Other come drive actuator do not have a fixed based, but have two moving members supported by an insulated spring-like material. The insulated spring-like material causes the comb drive actuators members to move away from each other when power is no longer applied to each member. A signal can be used to causes comb drive actuators to move into and away from each other in accordance with the signal.

Referring to FIG. 5, an electromechanical device in accordance with features of the present invention is illustrated. The device includes several comb drive actuators 30 assembled together forming a chain similar to that formed by link in a wrist-watch band. Each comb drive actuator is designed to have a contact areas and a set of opposite facing teeth, which makes formation of a chain possible. As shown in the drawings, electrical power is staggered along the chain so that positive voltage 31 is applied to every other comb drive, while negative (or common) electrical contact is applied to the non-positive comb drives. When electrical power is applied to the chain of comb drives, the chain shortens because of the attraction caused by the electrified teeth. The teeth can be insulated using a wear resistant coating. The coating will prevent shorting between comb drive elements 30.

Referring to FIG. 6, the comb drive actuators 30 forming the chain described in FIG. 4 are shown surrounded by a tube-like structure 35. The tube-like structure is an outer, insulative coating 35 for the electromechanical contacts (e.g., comb drives). The coating 35 is flexible and compressible and should prevent the electromechanical hardware (e.g., comb drives 30) from interfering with the heart or other internal organs or tissue. The coating 35 also prevents the system from shorting from exposure to bodily fluids. The coating is made of a material (e.g. Gortex™) that is commonly used in surgical procedures with a purpose for lasting long durations in the body. The coating 35 cannot be easily rejected by the body and must be able to assimilate to the internal environment of the human body for relatively long periods of time.

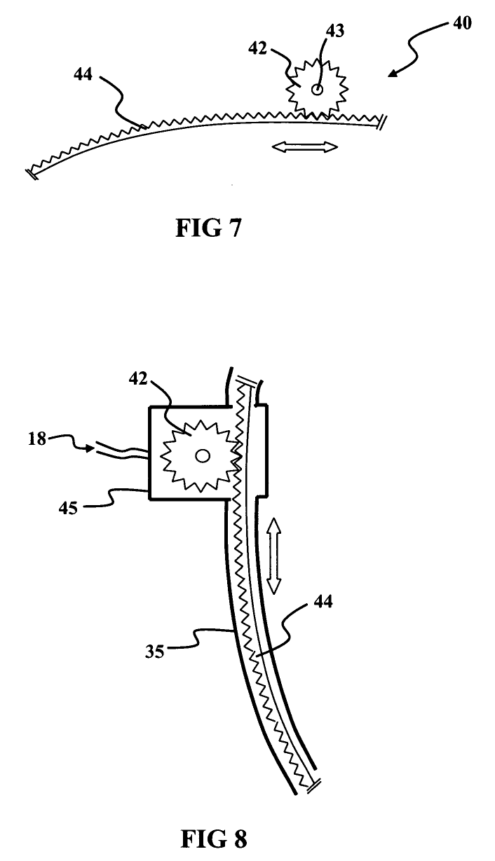

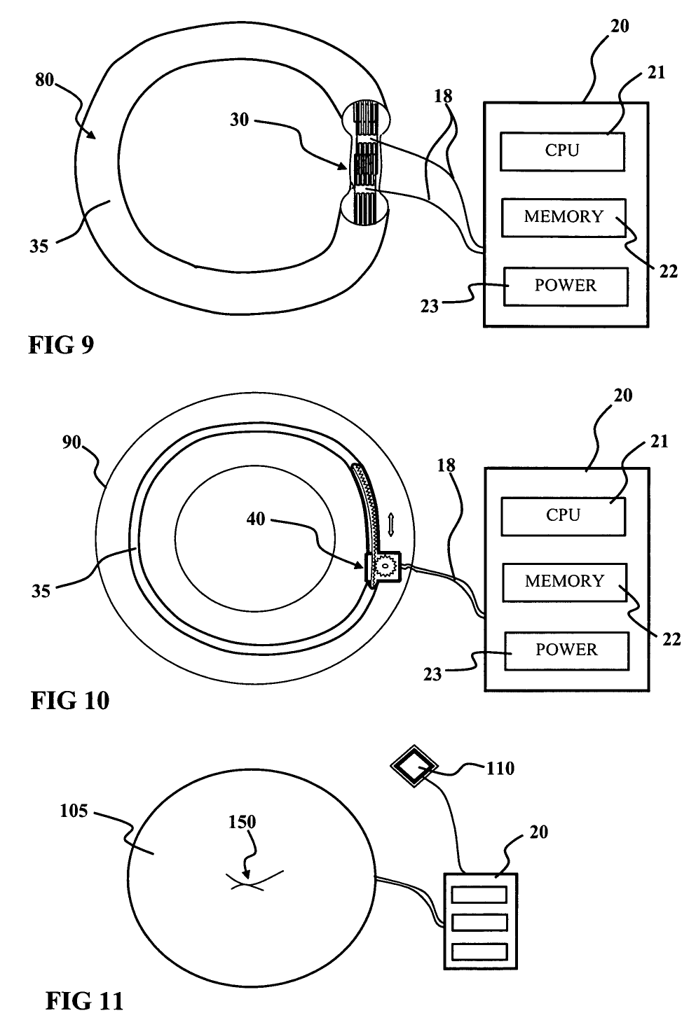

Referring to FIG. 7, another electromechanical system 40 is shown for operation in accordance with an embodiment. The electromechanical system 40 includes a gear 42 having teeth and rotating on a hub 43, and a strap 44 also with teeth that are complimentary to the gear 42 teeth. When the gear spins, the strap 44 moves along the gear 42, which is commonly, understood mechanics. Referring to FIG. 8, however, the gear 42 and strap 44 are shown enclosed within a protective housing 45 and tube 35, respectively. The tubular material 35 is similar to that described in FIG. 5 for the comb drive system 30. The housing 45 protects the gear from bodily fluids, and also protects the body from mechanical movement. Electrical wires 18 are shown coupled to the housing. The wires provide power to the gear 42.

Referring to FIG. 8, shown is a donut-shaped device 50, which operates as a biological valve, such as a sphincter valve. The valve can be made of the tubular material 35 that has been described previously. The valve is shown containing the comb drive system 30. The comb drive system is wired 18 to a controller 20. Referring to FIG. 10, another valve 90 is shown. This time, the valve 90 is shown as a separate unit containing the tubular material 35, which further contains the strap 44 of the gear tooth device 40. The housing is shown coupled to the tubular material wherein the strap 44 can be moved using the gear and become shortened or loosened. In order for the bio valve to remain in a closed position, the insulating material can posses elastic properties that maintain the bio valve in closed position until power applied to the electromechanical system forces the bio valve open. By providing material that keeps the bio valve in a normally closed position, power will not be required until the bio valve requires opening. The electromechanical systems can also be adapted with springs or magnetic force to cause the bio valve to remain closed until power is applied.

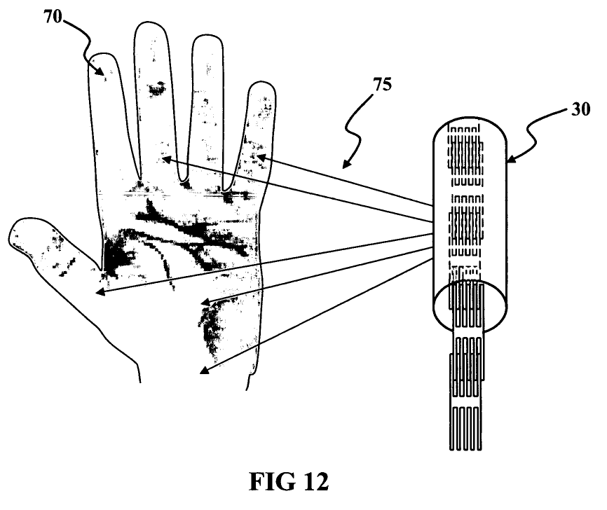

Referring to FIG. 11, a bio valve 105 is shown in a closed position 150. Also shown associated with the bio valve 105 are a controller 20 and a sensor 110. The sensor 110 and controller 20 can be programmed to cause the bio valve 105 to open or closed in accordance with a specific application. Closure of the valve 105 is caused when an electromechanical system contained by inside the tubular shape of the valve is caused to tighten, thereby causing the valve 105 to close. The valve 105 can be opened when the electromehanical system is allowed to release (e.g., comb drive system 30) or reverse movement (e.g., gear drive system 40). For example, if the valve 105 is being used as the sphincter valve between the esophagus and the stomach, then GERD can be prevented when a patient is not eating.

When a sensor located above the sphincter valve 105 is activated because it senses food traveling into the esophagus, then the valve is cause to relax or open. The sensor can be a pressure transducer, electrical contact sensor, or electro-impulse detector. A pressure transducer can sense the weight of food or water within the esophagus above the valve. It can now be appreciated that a similar sensor-valve configuration can be employed in other parts of the human body. For example, the sphincter valve 105 can be implanted in a patient's rectum or after the bladder. The valve can help patient control incontinence. Such an application would be helpful for cancer patients that have lost functionality due to rectum or prostrate cancer, or adults that can no longer control urinary function because of age or numerous childbirths.

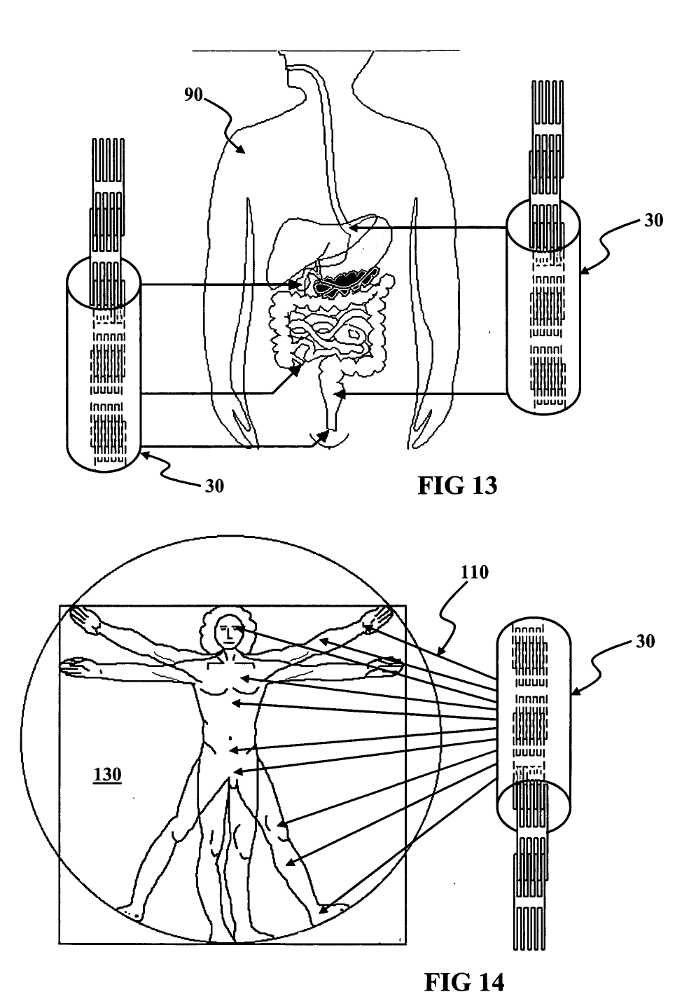

FIG. 12 illustrates a hand 70 with arrows 75 pointing from an electromechanical system 30 to areas on the hand where mechanical function may be of help. Tendons in hands, feet, arms legs, etc., may no longer function well because of arthritis or because of nerve loss. IT can now be appreciated following this description that electromechanical systems can be devices to assist in the movement of tendons by muscles located within a body's extremities. Referring to FIG. 13, a patient 90 is shown with arrow pointing to areas within the digestive tracts wherein electromechanical systems 30 may assist with control functions. Referring to FIG. 14, a human body 130 is shown with arrows 110 pointing to location on the body where electromechanical systems 30 may assist with bodily movement.

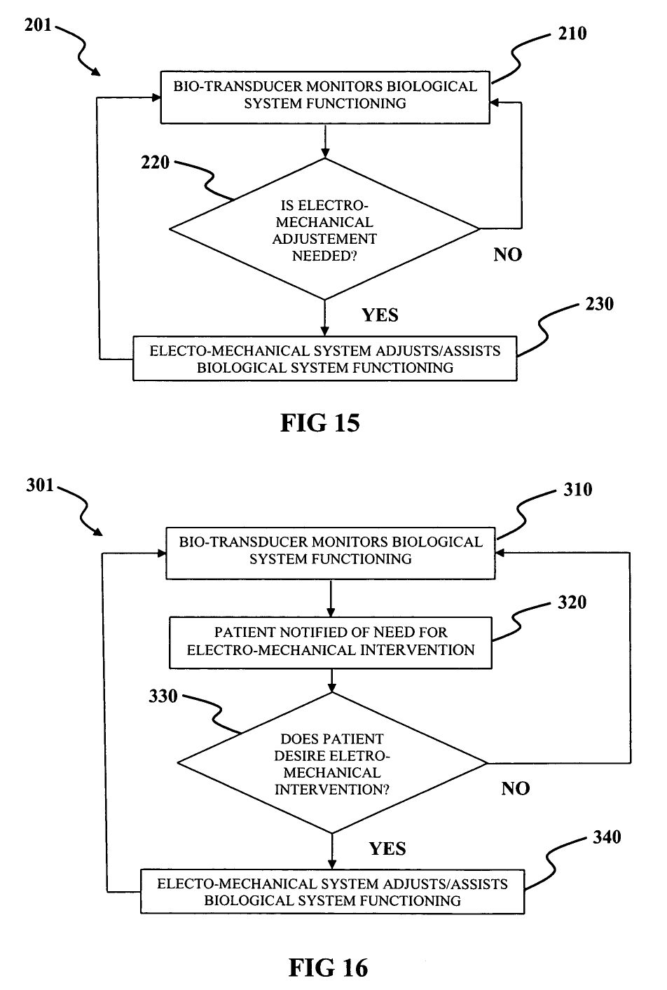

Referring to FIG. 15, a flow diagram 201 is shown including steps of electromechanical system function in the human body. A controller/monitor, similar to the controller 20 and sensors 15/110 previously described can carry out the following steps. As shown in block 210, a bio-transducer monitors biological system functioning. As shown in decision block 220, the system inquires whether electroemchanical adjustment is needed. If not, the process returns/maintains monitoring status of block 210. If adjustment is necessary, the as shown in block 230, an electromechanical system adjusts/assists a biological system with functioning. It can now be appreciated that the monitoring can cause operation where, for example, food is sensed in the esophagus, or when the heart requires faster/slower operation based on load requirements of the patients (e.g., exercise, or rest).

Referring to FIG. 16, a flow diagram 301 is shown where patient intervention can be allowed to a system. As shown in block 310, a bio transducer monitors a biological system's functioning. As shown in block 320, a patient can be notified of a need for electromechanical intervention. Notification can occur, for example, where the patient is exerting himself and requires faster pumping of the heart, or when a sensor indicates (e.g., vibrates, alarms, or other sensation) that a valve must be operated. As shown in decision block 330, the system is waiting for input by a patient as to whether electromechanical intervention is needed. If not, then monitoring continued in block 310. If intervention is requested, then the electromechanical system can cause adjustments or assistance of a biological system for occur as thought herein.

A controller 60 is generally in communication with said plurality of electromechanical elements 30/40, while a microprocessor 90 is generally in communication with controller 60. Microprocessor 90 and controller 60 can be implemented in the context of a pacemaker 90, which is generally in communication with electrical devices. Microprocessor 90 can be implemented as a central processing unit (CPU) on a single integrated circuit (IC) computer chip. Microprocessor 90 generally functions as the central processing unit of apparatus 70, and can interpret and execute instructions, and generally possesses the ability to fetch, decode, and execute instructions and to transfer information to and from other resources over a data-transfer path or bus.

Note that each electromechanical element among said plurality of electromechanical elements can contract toward one another in systole and away from one another by a reversal of poles in diastole. Additionally, each electromechanical element among said plurality of electromechanical elements will preferably sequentially contract the heart horizontally and thereafter, vertically. As indicated previously, each electromechanical element is electrical insulated. Sheet 10 can be configured from a flexible or pliable material. Tube 35 can be configured from a flexible or pliable material.

Unique features of the electromechanical-biological system (EBS) described herein includes: integrated wire network, sensory feedback, controlled contraction or relaxation of any single actuator or actuator groups, programmable contraction or expansion, reflexic contraction or expansion from natural internal pacemakers, programmable contraction and expansion of any regions and sub-regions, programmable response to stimulus, resistance to mechanical failure since multiple components operate in parallel or over a grid configuration.

As a cardiac patch, the present invention offers a simpler design than a whole-heart wrap design and can be used to target a specific location of failure along an organ. The cardiac patch can be surgically affixed to cardiac regions and surfaces along a heart. For example, a patch can be placed over area of myocardial scar, aneurysm, or defect. The patch is sutured in place over the afflicted area. The electromechanical system within the patch can be programmed to contract and expand with heart cycles that are being sensed using sensors located near or within the patch and monitored by a microprocessor. Using this configuration, sub regional contraction and expansion is optimized with external programming and radiologic real-time visualization. Other advantages of the patch system are that it provides self-contractile material to reinforce weakened or absent myocardium. The externally applied patch need not contact blood. Coagulation problems are avoided. Surgical excision of defective tissue is avoided.

Because artificial Anorectal Sphincters are desperately needed by fecal incontinence patients (stomates patients with a surgically removed rectum or anus and a diverting colostomy). An electromechanical system can be surgically implanted to surround native anorectum or surgically translocated conduit (colon pulled into place formerly occupied by the anorectum). Baseline conformation is relaxation of upstream canal and relative contraction of downstream canal. Manual switch activation or direct signal transduction from the sacral and inferior hemorrhoidal nerves allows defecation by stimulating upstream canal contraction and downstream canal relaxation. Reflex continence is maintained when the switch is not activated or by voluntary impulses. In these conditions, propagating impulses sensed from upstream bowel produce a reflex increased capacitance of the upstream sleeve and temporary hypercontraction of the downstream sleeve. A relatively thin artificial sphincter assist produces a programmable limit of pressure on tissue.

Now, an artificial Urinary Sphincter can be provided in accordance with feature of the present invention to prevent urinary Incontinence caused by female stress or side affects of male surgery for prostrate issues. An Artificial Gastroesophageal Sphincter provided utilizing features of the present invention can prevent gastroesophageal reflux. A cylindrical tube including electroemchanical functioning can be surgically implanted to fit around the gastroesophageal junction in a patient. Relatively contracted in baseline conformation to prevent gastroesophageal reflux. The Artificial Gastroesophageal Sphincter of the present invention is induced to relax by sensed distension of upstream esophagus. Anti-reflux prosthetic devices of the past (e.g., Angelchick prosthesis) can now be abandoned because of prior problems with prosthesis migration or erosion.

The embodiments and examples set forth herein are presented to best explain the present invention and its practical application and to thereby enable those skilled in the art to make and utilize the invention. Those skilled in the art, however, will recognize that the foregoing description and examples have been presented for the purpose of illustration and example only. Other variations and modifications of the present invention will be apparent to those of skill in the art, and it is the intent of the appended claims that such variations and modifications be covered.

The description as set forth is not intended to be exhaustive or to limit the scope of the invention. Many modifications and variations are possible in light of the above teaching without departing from the scope of the following claims. It is contemplated that the use of the present invention can involve components having different characteristics. It is intended that the scope of the present invention be defined by the claims appended hereto, giving full cognizance to equivalents in all respects.

The embodiments of the invention in which an exclusive property or right is claimed are defined as follows. Having thus described the invention what is claimed is:

1. A electromechanically-based biological system interface, comprising:
electromechanically actuated hardware;
a protective coating surrounding the eletromechanically actuated hardware and acting as a barrier between the electromechanically actuated hardware and biological systems;
at least one sensor to monitor biological system functions;
a microprocessor analyzing biological system functions measured by the at least one sensor;
a controller causing operation of the electromechanically actuated system to operate under direction of the microprocessor as at least one of: a ventricular assist device, bio valve, a muscle-tendon interface.

2. The system of claim 1 including the eletromechanically actuated hardware comprising more than one comb drive actuator assembled as at least one chain link wherein positive and ground connections are alternately connected to the more than one comb drive actuator forming the at least one chain link, wherein the chain link shortens as power is applied to the comb drive actuators and the comb drive expands when power is no longer alternately applied to the more than one comb drive actuator.

3. The system of claim 2 wherein more than one of said chain link is further assembled into a sheet-like grid and an integrated wire network provides sensory feedback, controlled contraction or relaxation of said more than one comb drive actuator.

4. The system of claim 3 wherein the controller is programmed to cause the electromechanically actuated hardware to cause contraction or expansion of a biological system.

5. The system of claim 4 wherein the contraction to expansion is of biological organs, artificial muscles, artificial valves.

6. The system of claim 3, wherein said sheet-like grid can be wrapped around a failing heart to support ventricular activities thereof.

7. The system of claim 1 including the eletromechanically actuated hardware comprising a gear including teeth on the outer perimeter thereof and located within a housing and a strap associated with the gear, said strap including teeth incorporated thereon that are complimentary to teeth on the gear, wherein the strap shortens as power applied to the gear causes the gear to turn and move the strap and the strap lengthens when power is no longer applied to the gear, causing the gear to rotate freely with movement of the strap.

8. The system of claim 7 wherein more than one set of said gear and associated strap is assembled into a sheet-like grid and an integrated wire network provides sensory feedback, controlled contraction or relaxation of said more than one set of said gear and associated strap.

9. The system of claim 8 wherein the controller is programmed to cause the electromechanically actuated hardware to cause contraction or expansion of a biological system.

10. The system of claim 9 wherein the contraction to expansion is of biological organs, artificial muscles, artificial valves.

11. The system of claim 8, wherein said sheet-like grid can be wrapped around a failing heart to support ventricular activities thereof.

12. The system of claim 2 wherein the at least one chain link is assembled into a circle and is surrounded by the protective coating, and the chain link formed in a circle is used as a bio valve adapted for use in a biological system to replace or supplement operation of a biological valve.

13. The system of claim 12 wherein said chain link assembled into a circle is used as a sphincter valve replacement within a human body.

14. The system of claim 7 wherein the gear and the strap associated with the gear are assembled into a circle and is surrounded by the protective coating, and the chain link formed in a circle is used as a bio valve adapted for use in a biological system to replace or supplement operation of a biological valve.

15. The system of claim 14 wherein the strap shortens as power applied to the gear causes the gear to turn and move the strap and the strap lengthens when power is no longer applied to the gear, causing the gear to rotate freely with movement of the strap and loosen the strap.

16. An apparatus for assisting biological system functions, the apparatus comprising:
a controller in communication with electromechanically actuated hardware; and
a protective coating surrounding eletromechanically actuated hardware and acting as a barrier between the electromechanically actuated hardware and biological systems.

17. The apparatus of claim 16 further comprising:
at least one sensor to monitor biological system functions; and
a microprocessor analyzing biological system functions measured by the at least one sensor.

18. The apparatus of claim 17, further comprising a controller, said controller causing operation of the electromechanically actuated system to operate under direction of the microprocessor as at least one of: a ventricular assist device, bio valve, a muscle-tendon interface.

19. The system of claim 16 wherein the eletromechanically actuated hardware comprises more than one comb drive actuator assembled as at least one chain link wherein positive and ground connections are alternately connected to the more than one comb drive actuator forming the at least one chain link, wherein the chain link shortens as power is applied to the comb drive actuators and the comb drive expands when power is no longer alternately applied to the more than one comb drive actuator.

20. The system of claim 19 wherein more than one of said chain link is further assembled into a sheet-like grid and an integrated wire network provides sensory feedback, controlled contraction or relaxation of said more than one comb drive actuator.

21. The system of claim 18 wherein the controller is programmed to cause the electromechanically actuated hardware to cause contraction or expansion of a biological system.

22. The system of claim 21 wherein the contraction to expansion is of biological organs, artificial muscles, artificial valves.

23. The system of claim 20, wherein said sheet-like grid can be wrapped around a failing heart to support ventricular activities thereof.

24. The system of claim 16 including the eletromechanically actuated hardware comprising a gear including teeth on the outer perimeter thereof and located within a housing and a strap associated with the gear, said strap including teeth incorporated thereon that are complimentary to teeth on the gear, wherein the strap shortens as power applied to the gear causes the gear to turn and move the strap and the strap lengthens when power is no longer applied to the gear, causing the gear to rotate freely with movement of the strap.

25. The system of claim 24 wherein more than one set of said gear and associated strap is assembled into a sheet-like grid and an integrated wire network provides sensory feedback, controlled contraction or relaxation of said more than one set of said gear and associated strap.

26. The system of claim 19 wherein the at least one chain link is assembled into a circle and is surrounded by the protective coating, and the chain link formed in a circle is used as a bio valve adapted for use in a biological system to replace or supplement operation of a biological valve.

27. The system of claim 26 wherein said chain link assembled into a circle is used as a sphincter valve replacement within a human body.

28. The system of claim 24 wherein the gear and the strap associated with the gear are assembled into a circle and is surrounded by the protective coating, and the chain link formed in a circle is used as a bio valve adapted for use in a biological system to replace or supplement operation of a biological valve.

29. The system of claim 28 wherein the strap shortens as power applied to the gear causes the gear to turn and move the strap and the strap lengthens when power is no longer applied to the gear, causing the gear to rotate freely with movement of the strap and loosen the strap.

30. A electromechanically-based biological system interface, comprising:
electromechanically actuated hardware;
a protective coating surrounding the eletromechanically actuated hardware and acting as a barrier between the electromechanically actuated hardware and biological systems; and
a microprocessor and controller causing the electromechanically actuated system to operate as at least one of: a ventricular assist device, bio valve, a muscle-tendon interface.

31. The system of claim 30 including the eletromechanically actuated hardware comprising more than one comb drive actuator assembled as at least one chain link wherein positive and ground connections are alternately connected to the more than one comb drive actuator forming the at least one chain link, wherein the chain link shortens as power is applied to the comb drive actuators and the comb drive expands when power is no longer alternately applied to the more than one comb drive actuator.

32. The system of claim 31 wherein more than one of said chain link is further assembled into a sheet-like grid and an integrated wire network provides sensory feedback, controlled contraction or relaxation of said more than one comb drive actuator.

33. The system of claim 30 wherein microprocessor and controller are programmed to cause the electromechanically actuated hardware to cause contraction or expansion of at least one of a heart or a sphincter valve.

34. The system of claim 32, wherein said sheet-like grid can be wrapped around a failing heart to support ventricular activities thereof and wherein the microprocessor and controller cause the sheet-like grid to cause contraction or expansion of a heart.

35. The system of claim 30 including the eletromechanically actuated hardware comprising a gear including teeth on the outer perimeter thereof and located within a housing and a strap associated with the gear, said strap including teeth incorporated thereon that are complimentary to teeth on the gear, wherein the strap shortens as power applied to the gear causes the gear to turn and move the strap and the strap lengthens when power is no longer applied to the gear, causing the gear to rotate freely with movement of the strap.

36. The system of claim 35 wherein more than one set of said gear and associated strap is assembled into a sheet-like grid and an integrated wire network provides sensory feedback, controlled contraction or relaxation of said more than one set of said gear and associated strap.

37. The system of claim 35 wherein the at least one chain link is assembled into a circle and is surrounded by the protective coating, and the chain link formed in a circle is used as a bio valve adapted for use in a biological system to replace or supplement operation of a biological valve.

38. The system of claim 37 wherein said chain link assembled into a circle is used as a sphincter valve replacement within a human body.

39. The system of claim 37 wherein the strap shortens as power applied to the gear causes the gear to turn and move the strap and the strap lengthens when power is no longer applied to the gear, causing the gear to rotate freely with movement of the strap and loosen the strap.
